(19) United States
(12) Patent Application Publication
(10) Pub. No.: US 2006/0224004 A1
(43) Pub. Date: Oct. 5, 2006

(54) Process for preparing ursodeoxycholic acid di-sodium 3,7-disulfate

(76) Inventors: Massimo Ferrari, Cenate Sotto (IT); Matteo Bonaldi, Chiuduno (IT); Fabrizio Zinetti, Casazza (IT)

Correspondence Address: Jay S Cinamon;Abelman Frayne & Schwab, 10th Floor, 666 Third Avenue, New York, NY, 10017-5621, US

(21) Appl. No.: 10/553,582

(22) PCT Filed: Apr. 16, 2004
(86) PCT No.: PCT/EP04/50538

§ 371 Date: Oct. 17, 2005

(30) Foreign Application Priority Data
Apr. 18, 2003 (IT) MI2003A000822

(51) Int. Cl. C07J 41/00

(52) U.S. Cl. 552/550

(57) ABSTRACT

Process for preparing ursodeoxycholic acid di-sodium 3,7-disulfate comprising: a) reacting ursodeoxycholic acid with sulfamic acid to give ursodeoxycholic acid di-ammonium 3,7-disulfate; b) treating ursodeoxycholic acid di-ammonium 3,7-disulfate with organic sodium bases or inorganic sodium bases and then treating the reaction mixture with an inorganic acid until a pH between 3.0 and 4.5 is reached to give ursodeoxycholic acid di-sodium 3,7-disulfate in solution; c) removing the salts present (by filtration) and then precipitating ursodeoxycholic acid di-sodium 3,7-disulfate by diluting with an organic solvent.

### FIELD OF THE INVENTION

The present invention relates to a process for preparing ursodeoxycholic acid di-sodium 3,7-disulfate.

### PRIOR ART

Ursodeoxycholic acid (UDCA) is a well-known product (as described in The Merck Index, 12^{th} edition) and likewise known is its hepato-protective activity, which makes UDCA an effective drug for qualitative and quantitative alterations in biligenetic function, also used in painful dyspeptic symptomology due to calculous or acalculous cholecystopathy, in dyskinesia of the biliary ducts and associated syndromes and in lipemic alterations due to increased cholesterol and/or triglycerides. UDCA is practically insoluble in water, a characteristic which limits its therapeutic use to solid oral forms only such as capsules and tablets.

Also described in the art are derivatives of UDCA which, while maintaining the aforesaid therapeutic qualities thereof, being soluble in water, can be administered in a different manner and with greater efficacy.

Among these forms derived from ursodeoxycholic acid, of particular interest is ursodeoxycholic sulfate acid, and the corresponding sodium salts as described in patents EP 117570 and U.S. Pat. No. 5,763,435.

These documents describe syntheses which propose the use of dangerous and toxic reagents, such as the pyridine-sulfuric anhydride complex or chlorosulfonic acid and pyridine, combined, in the latter instance, with complicated and laborious processing stages, in particular because of the specific plant technology necessary for separating the process intermediate using absorbent cartridges.

The need was therefore felt for a process for preparing ursodeoxycholic acid sodium 3,7-disulfate capable of overcoming the safety and toxicity problems connected with the use of the necessary reactants described in the art, and of avoiding plant related problems due to separating the process intermediate using absorbent cartridges.

### SUMMARY OF THE INVENTION

A new process has now been found for preparing ursodeoxcycholic acid di-sodium 3,7-disulfate from ursodeoxycholic acid, able to overcome the technical drawbacks and those connected with the need to use reactants which are dangerous and toxic to handle, pertaining to the known processes of the art.

The Applicant has unexpectedly and surprisingly found a new process for preparing ursodeoxycholic acid sodium 3,7-disulfate from ursodeoxycholic acid by means of sulfamic acid comprising the formation of ursodeoxycholic acid di-ammonium 3,7-disulfate as reaction intermediate. The process of the invention is characterised by having few stages with easy industrial applicability and high yield, starting from reagents easily obtainable commercially, which have low cost and are safer in their handling compared with raw materials indicated in the known art.

### DESCRIPTION OF THE FIGURES

FIG. 1: synthetic route of preparation of ursodeoxycholic acid sodium 3,7-disulfate

FIG. 2: shows the IR spectrum of ursodeoxycholic acid di-ammonium 3,7-disulfate

FIGS. 3-8: show the diagrams and mass spectra of ursodeoxycholic acid di-ammonium 3,7-disulfate.

FIGS. 9-10: show the ¹H-NMR spectra of ursodeoxycholic acid di-ammonium 3,7-disulfate in DMSO-d₆ solvent.

FIG. 11: shows the ¹H-NMR spectrum of ursodeoxycholic acid di-ammonium 3,7-disulfate in DMSO-d₆ solvent+D₂O.

FIG. 12: shows the ion trap mass spectrum using spectrometric analysis by infusion of ursodeoxycholic acid di-ammonium 3,7-disulfate.

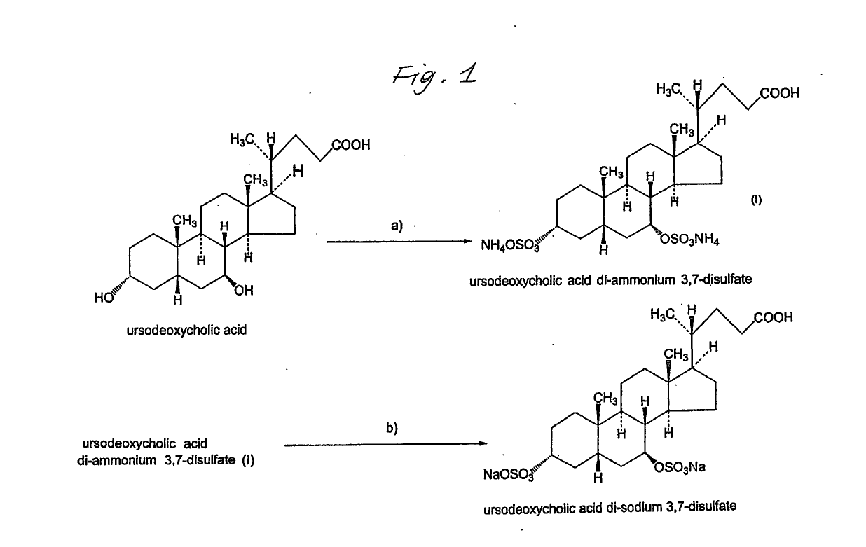

### DETAILED DESCRIPTION OF THE INVENTION

An aspect of the present invention therefore is a process for preparing ursodeoxycholic acid di-sodium 3,7-disulfate comprising:

a) reacting ursodeoxycholic acid with sulfamic acid thereby obtaining ursodeoxycholic acid di-ammonium 3,7-disulfate;

b) treating ursodeoxycholic acid di-ammonium 3,7-disulfate with organic sodium bases or inorganic sodium bases and then treating the reaction mixture with an inorganic acid until a pH between 3.0 and 4.5 is reached thereby obtaining ursodeoxycholic acid di-sodium 3,7-disulfate in solution.

The reaction of stage a) is preferably conducted in an aprotic solvent, more preferably in N,N-dimethylformamide, at a temperature between 40° C. and 110° C., preferably between 80° C. and 90° C.

From the reaction mixture of stage a) ursodeoxycholic acid di-ammonium 3,7-disulfate is separated by means of techniques well known in the field for recovering products in solution, preferably by fractional crystallisation with acetone.

In stage b) the inorganic sodium bases are preferably chosen from the group consisting of: sodium hydroxide, sodium carbonate and sodium bicarbonate, more preferably sodium hydroxide is used; the organic sodium bases are sodium acetate.

In stage b) the treatment of ursodeoxycholic acid di-ammonium 3,7-disulfate with organic sodium bases or inorganic sodium bases is carried out in an alcoholic solvent, preferably linear or branched lower C1-C4 alcohols, more preferably methanol and its mixtures with other organic solvents (for example acetone).

The treatment with organic sodium bases or inorganic sodium bases is preferably conducted at a temperature between −10° C. and 30° C., more preferably between 0 and 5° C., operating under vacuum to favour the removal of the ammonia released.

The subsequent acidification of the reaction mixture after treatment with organic sodium bases or inorganic sodium bases, with the purpose of adjusting the pH to values between 3.0 and 4.5, preferably between 3.0 and 4.0, is achieved by treating the reaction mass with an inorganic acid preferably chosen from the group consisting of: hydrochloric acid, sulphuric acid, 85% (w/w) phosphoric acid or their mixtures, preferably with 85% phosphoric acid.

One of the particularly preferred embodiments of the process for preparing ursodeoxycholic acid di-sodium 3,7-disulfate, comprises, in addition to aforesaid stages a) and b), a stage c), relating to the recovery of ursodeoxycholic acid di-sodium 3,7-disulfate from the reaction mixture, consisting of: c′) removing, by filtration, the precipitated inorganic salts formed after acidification treatment and c″) precipitating ursodeoxycholic acid di-sodium 3,7-disulfate from the filtrate whereby the solution containing ursodeoxycholic acid di-sodium 3,7-disulfate is concentrated by distillation and the residue is re-dissolved in an organic solvent, preferably acetone, to isolate ursodeoxycholic acid di-sodium 3,7-disulfate.

In stage c′) the filtration to remove the precipitated inorganic salts formed after acidification treatment is facilitated by treating the reaction mixture derived from stage b) with organic solvents, preferably acetone.

In stage c″) the residue obtained by concentrating the solution containing ursodeoxycholic acid di-sodium 3,7-disulfate by distillation is re-dissolved in organic solvents or water or mixtures of solvents and water, acetone being preferably used, at a temperature between 20° C. and 70° C., more preferably between 55 and 65° C., the suspension thus obtained then being cooled to room temperature and filtered to obtain ursodeoxycholic acid di-sodium 3,7-disulfate as precipitate.

A further aspect of the present invention is ursodeoxycholic acid di-ammonium 3,7-disulfate of formula: 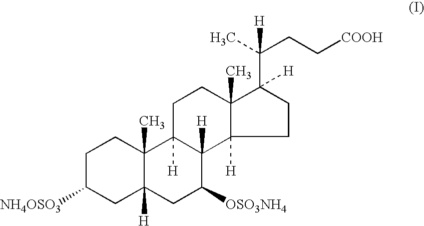
obtained as intermediate in the preparation process of ursodeoxycholic acid di-sodium 3,7-disulfate, and the process for synthesising ursodeoxycholic acid di-ammonium 3,7-disulfate, comprising reacting ursodeoxycholic acid with sulfamic acid, preferably in an aprotic solvent, more preferably N,N-dimethylformamide, at a temperature between 40° C. and 110° C., preferably between 80° C. and 90° C.

According to a particularly preferred embodiment of the present invention, ursodeoxycholic acid in N,N-dimethylformamide, heated initially to 40-60° C., preferably to 45-50° C. is reacted with sulfamic acid at a temperature between 80 and 90° C.

The reaction mixture is cooled to a temperature between 35 and 50° C., preferably between 40-45° C., an organic solvent preferably acetone is added and the mass further cooled, and the ursodeoxycholic acid di-ammonium 3,7-disulfate is separated by filtration, being the intermediate in the preparation process for ursodeoxycholic acid di-sodium 3,7-disulphate.

The ursodeoxycholic acid di-ammonium 3,7-disulfate thus obtained is added while stirring to a solution of sodium hydroxide in alkyl alcohol, in this instance methanol, at a temperature between 0 and 5° C., placing the entire reaction mixture in a reactor under vacuum to facilitate the removal of the ammonia released. The pH of the reaction mixture is adjusted to between 3.0 and 4.5, preferably between 3.0 and 4.0, by adding an inorganic acid preferably chosen from the group consisting of hydrochloric acid, sulphuric acid, 85% (w/w) phosphoric acid or their mixtures, more preferably 85% phosphoric acid.

Precipitation of inorganic salts takes place while ursodeoxycholic acid di-sodium 3,7-disulfate remains in solution.

The suspension obtained, to which an organic solvent, preferably acetone, is added, is filtered, recovering from one side a solution containing ursodeoxycholic acid di-sodium 3,7-disulfate and inorganic salts from the other side.

After possible further dilution with deionised water, the solution containing ursodeoxycholic acid di-sodium 3,7-disulfate is concentrated by distillation until precipitation begins and the residue is re-dissolved in an inorganic solvent, preferably acetone, preferably at a temperature between 20° C. and 70° C., more preferably between 55° C. and 65° C., and the suspension thus obtained is then cooled to room temperature and filtered to obtain ursodeoxycholic acid di-sodium 3,7-disulfate as precipitate.

As shown by tests carried out by the Applicant, the process of the present invention enables ursodeoxycholic acid di-sodium 3,7-disulfate to be obtained by means of a few easily achievable reactions characterised by yields between more than 70% and 90%, using easily available solvents and reagents, under mild and easily controlled reaction conditions, starting from easily available base reactants such as ursodeoxycholic acid and sulfamic acid.

Some illustrative but non-limiting examples of the present invention are given hereinafter.

The products and reaction intermediates were characterised using the analysis techniques of FT-IR, HPLC/Mass Spectrometry (MS), differential scanning calorimetry (DSC), NMR: ¹H, ion trap spectrometry and ammonium ion selective electrode, together with elemental analysis of ursodeoxycholic acid di-sodium 3,7-disulfate.

The analytical data given hereinafter were obtained under the following conditions:
IR spectra with FT-IR technique obtained from samples in KBr tablets;
NMR spectra recorded on samples dissolved in DMSO-d₆ or DMSO-d₆+D₂O;
Mass spectra (MS) with HPLC/MS technique obtained by electron impact at 70 eV;
Mass spectra by infusion;
Spectra/thermograms obtained at a scan rate of 10° C./min within the temperature range: 100° C.-320° C.

### EXAMPLE 1

### Preparation of ursodeoxycholic acid di-ammonium 3,7-disulfate

50 g of ursodeoxycholic acid (M.W.: 392.58, equal to 0.127 moles) and 75 g of N, N-dimethylformamide are fed into a reactor. The mass is heated to 45-50° C., then 27 g of sulfamic acid (M.W.: 97.1, equal to 0.278 moles) are added. The reaction mass is heated to 80°-90° C. for thirty minutes, is then cooled to 40-45° C. and 400 g of acetone are added. The precipitated mass is stirred at 20-30° C., then the solid is filtered off and washed with 75 g of acetone. 70 g of ursodeoxycholic acid di-ammonium 3,7-disulfate are obtained, equal to a stoichiometric yield of 93.7%, based on the quantity of ursodeoxycholic acid.

The ursodeoxycholic acid di-ammonium 3,7-disulfate obtained and separated was identified and characterised as follows:

Analytical Data:

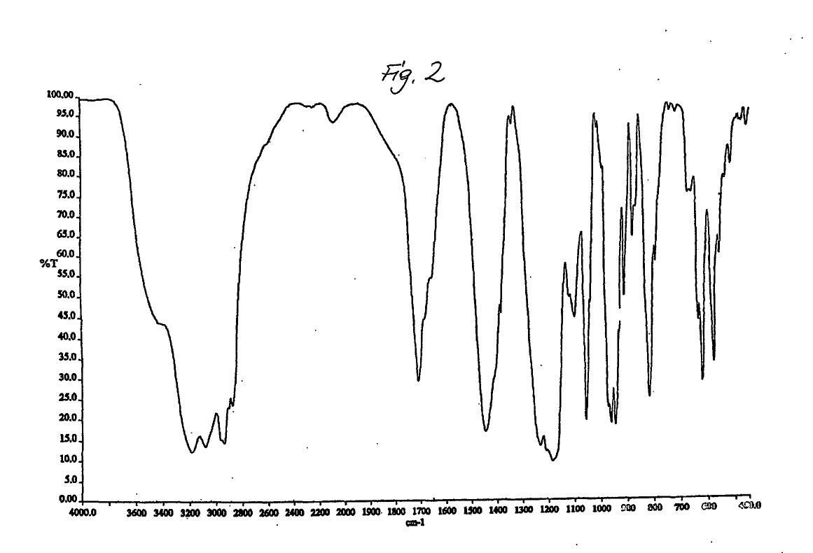

FT-IR analysis: 3500-2600 (O—H and NH₄⁺), 1711 (C═O), 1220-1187 (O—SO₂—O) 1/cm; the FT-IR spectrum is shown in FIG. 2;

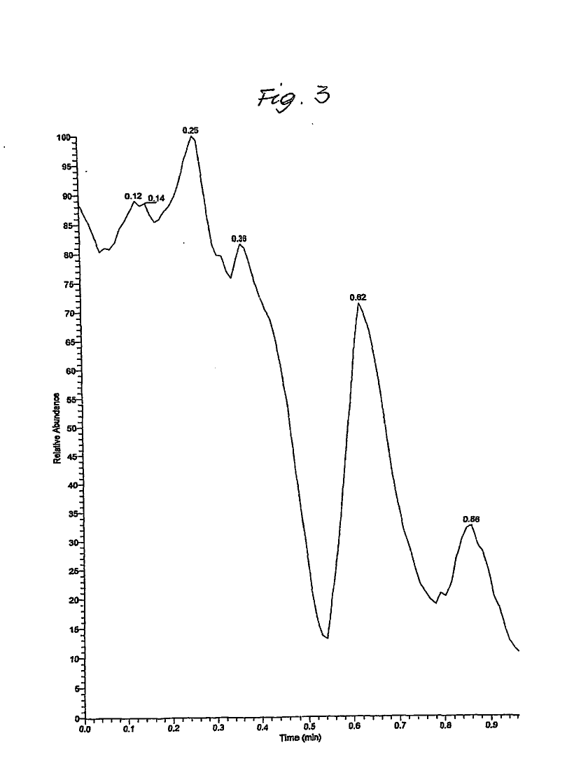

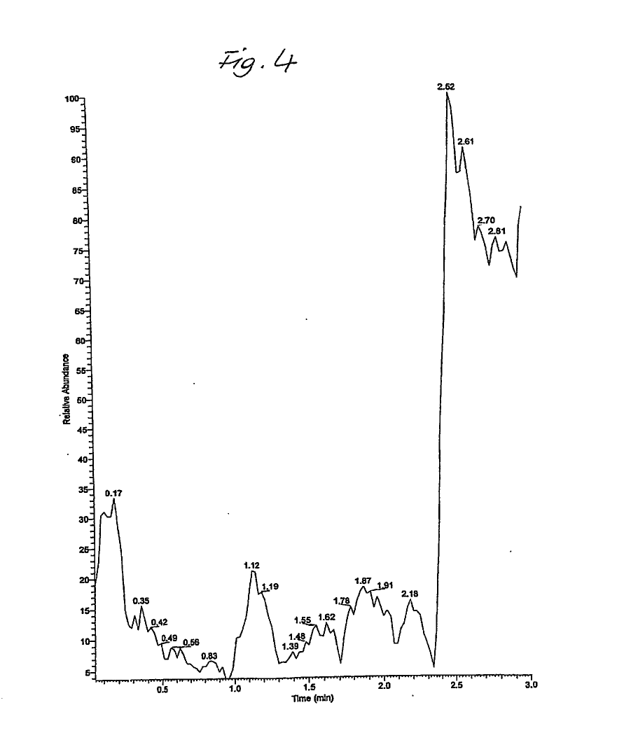

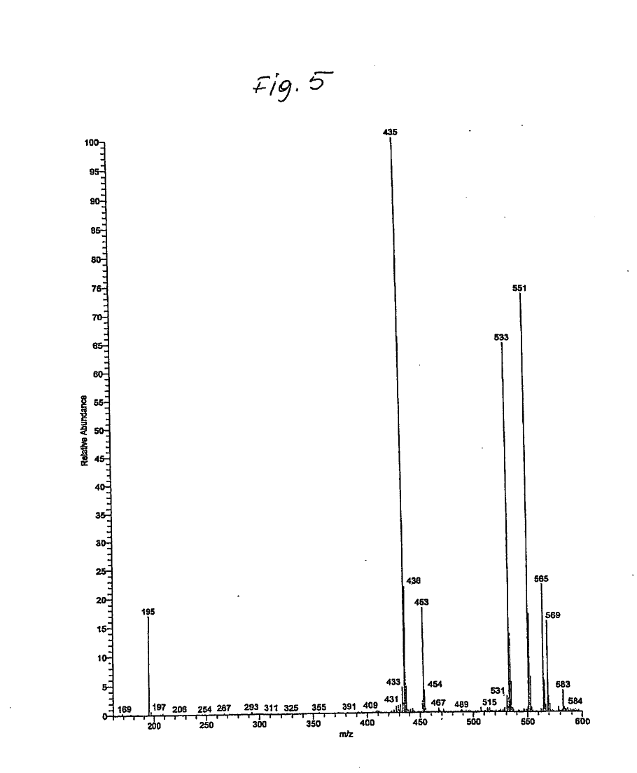

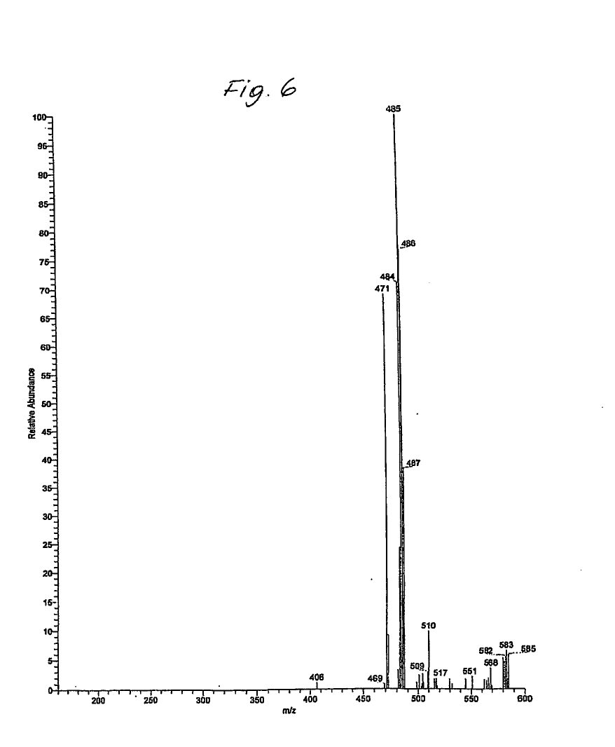

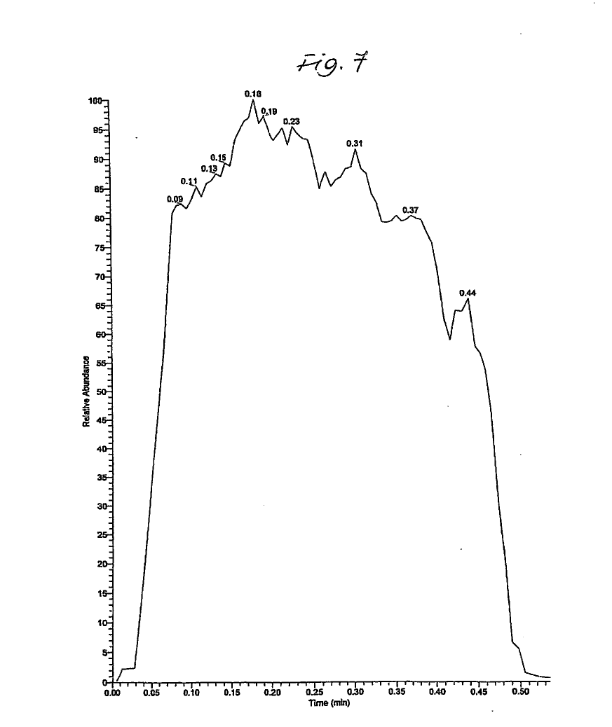

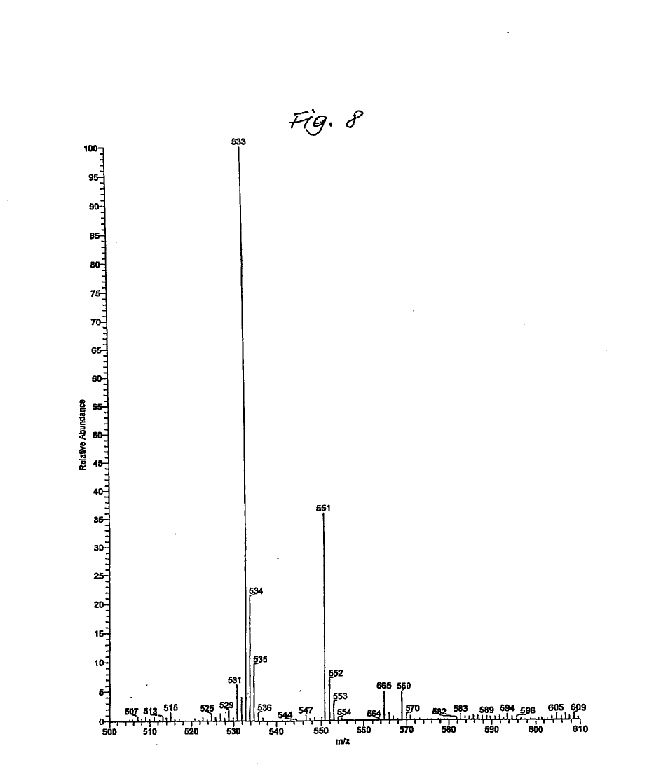

HPLC/MS analysis (m/z): 551, attribution [M-2NH₄⁺], with M: 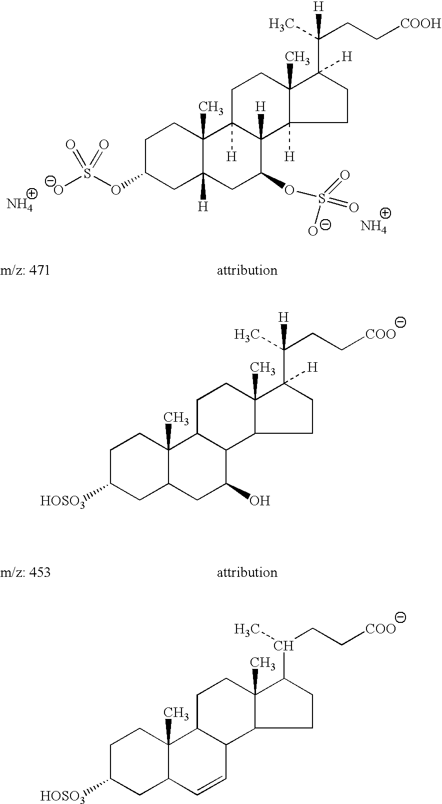
the HPLC/MS spectra are shown in FIGS. 3-7.

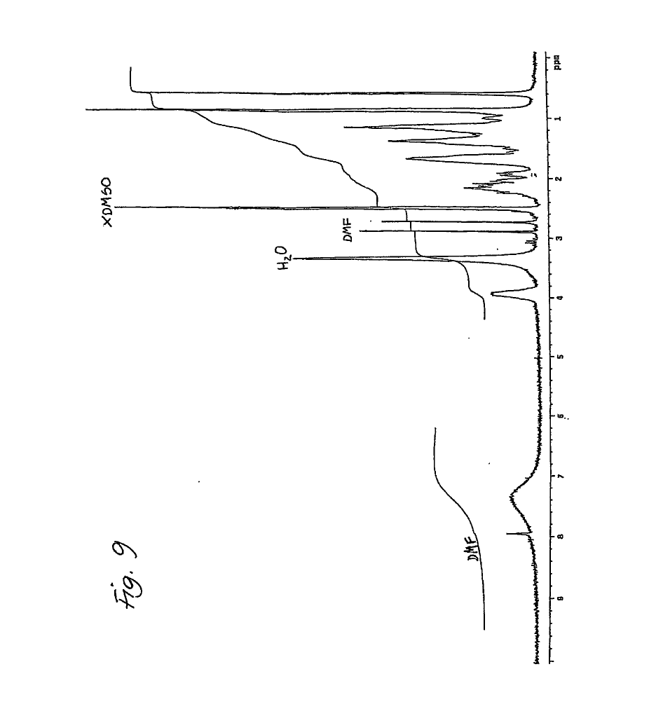

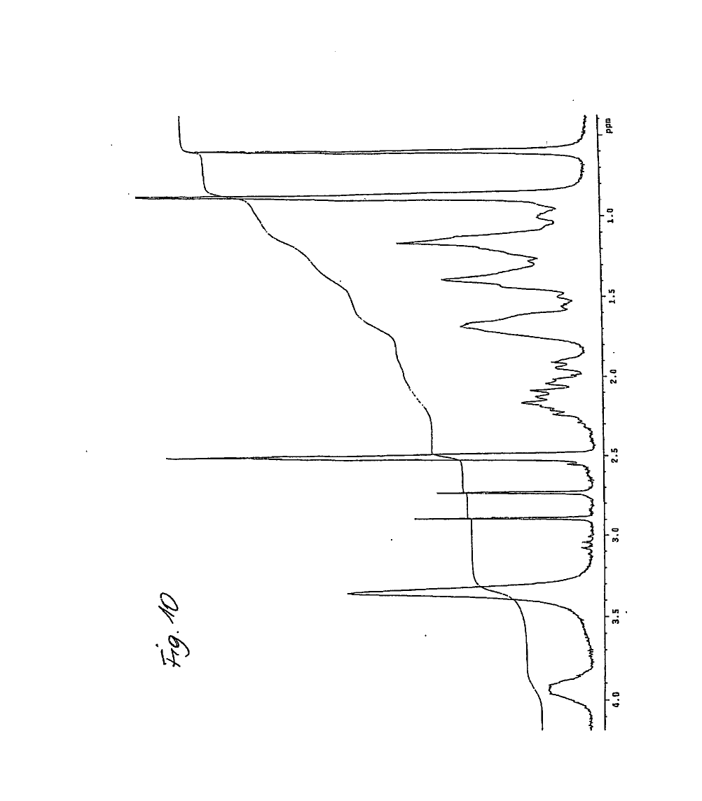

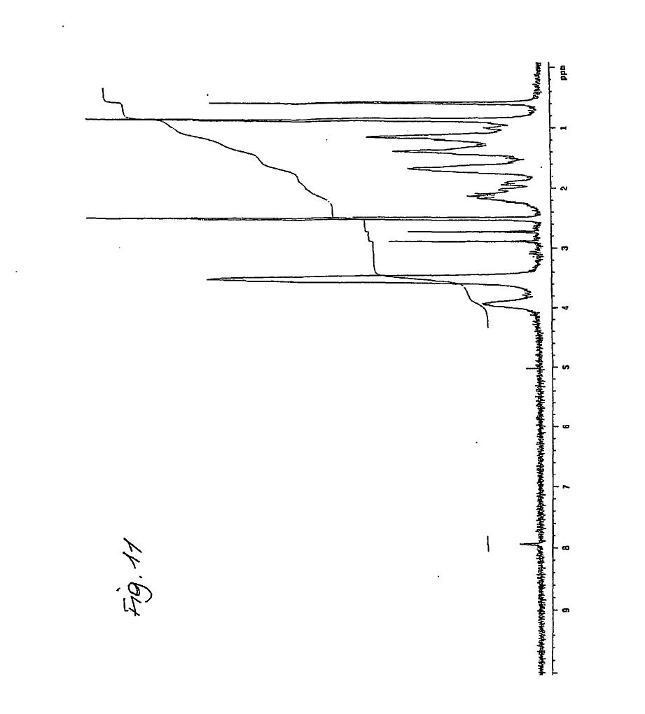

¹H-NMR analysis (200 MHz) in ppm: δ=0.6 ppm (3H s 21-CH3); δ=0.88 ppm (6H two s 18-CH₃, 19-CH₃); δ=1.01-2.24 ppm (26H m all other aliphatic H); δ=3.94 ppm (2H m 3-CH, 7-CH); δ=6.9-8.1 ppm (9H m widened OH and “exch.” NH4, in the presence of D₂O). The analysis has identified the presence of N,N-dimethyl-formamide. The ¹H-NMR spectra are shown in FIGS. 9-11.

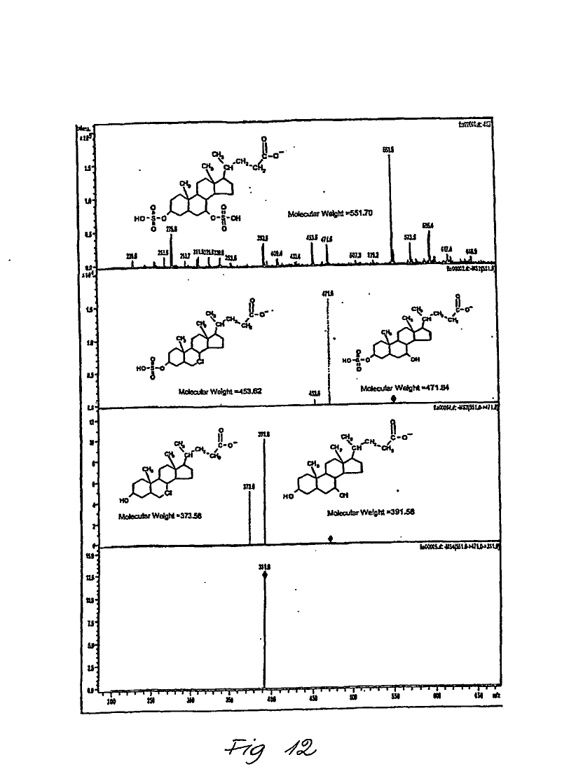

Mass spectrophotometric analysis by infusion: the presence of the dissociated adduct is evident i.e. does not contain the two ammonium groups; the identity of this adduct is also confirmed by the two successive fragmentations (MS2 and MS3). Beyond MS3 the compound is not subjected to further fragmentations. The spectra are shown in FIG. 12.

Ammonium ion selective electrode analysis: in the sample a nitrogen (ammoniacal) content equal to 4.76% was found. As the theoretical nitrogen content of the compound ursodeoxycholic acid di-ammonium 3,7-disulfate is 4.77%, this is a further confirmation of the structure:

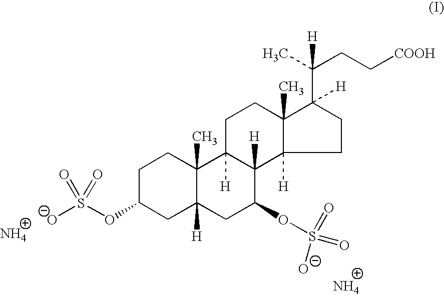

### Example 2

### Preparation of ursodeoxycholic acid di-sodium 3,7-disulfate

280 g of methanol and 19.3 g of sodium hydroxide (M.W.: 40, equal to 0.48 moles) are fed into a reactor. The mixture is stirred until dissolution is complete and 70 g of ursodeoxycholic acid di-ammonium 3,7-disulfate (M.W.: 586.68, equal to 0.12 moles) obtained as in example 1 are added at 0-5° C. The solution is stirred for thirty minutes at 0-5° C., placing the reactor under vacuum to remove the ammonia released, then the reaction mass is adjusted to pH 3.0-4.0 by adding in about 35 g of 85% phosphoric acid dropwise (in this way the inorganic salts precipitate, while the ursodeoxycholic acid di-sodium 3,7-disulfate remains in solution). 140 g of acetone are then added to the reaction mixture and the precipitated salts are filtered off and removed.

The solution containing the ursodeoxycholic acid di-sodium 3,7-disulfate thus obtained is fed into a reactor and after adding 25 g of deionised water, is concentrated by distillation until a thick residue is obtained. Finally, while maintaining the temperature of the mass at 55-65° C., 350 g of acetone are poured in. The precipitated mass is cooled to room temperature and the product is filtered off, washed with 70 g of acetone and dried at 80-90° C. 55 g of ursodeoxycholic acid di-sodium 3,7-disulfate are obtained equal to a stoichiometric yield of 77.2%, based on the quantity of ursodeoxycholic acid di-ammonium 3,7-disulfate.

The ursodeoxycholic acid di-sodium 3,7-disulfate obtained and separated was identified and characterised as follows:

Analytical Data:

FT-IR analysis=3488 (O—H), 2943-2873 (Cₛₚ³—H), 1711 (C═O), 1205-1211 (O—SO₂—O) 1/cm;

HPLC/MS analysis (m/z): 356, attribution [M-2(HOSO₃Na)]⁺; m/z: 341 attribution

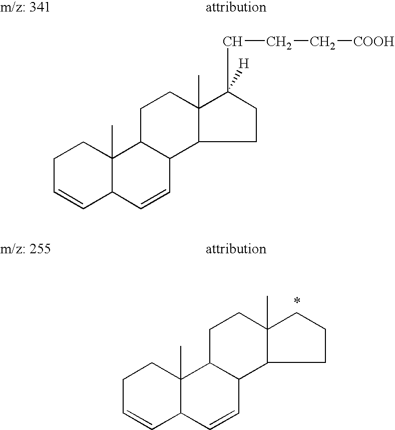

¹H-NMR analysis (200 MHz) in ppm: δ=0.6 ppm (3H s 21-CH₃); δ=0,88 ppm (6H two s 18-CH₃, 19-CH₃); δ=0.86-2.16 ppm (26H m all other aliphatic H); δ=3.92 ppm (2H m 3-CH, 7-CH); δ=11.0-13.0 ppm (1H s widened mobile OH).

DSC analysis: description of the thermogram indicating thermal events:

an endotherm with a peak at 167.16° C. corresponding to fusion, followed by an exotherm with a peak at 171.6° C. due to probable crystallisation, there follows:

an exotherm with a peak at 176.83° C. due to fusion of another polymorph;

an exotherm with a peak at 272.63° C. due to decomposition.

Elemental Analysis:

| | % Calculated | % Calculated for | |
|---|---|---|---|
| Element | for C₂₄H₃₈Na₂O₁₀S₂ | C₂₄H₃₈Na₂O₁₀S₂.0.7H2O | % Found |
| C | 48.31 | 47.31 | 46.98 |
| H | 6.42 | 6.51 | 6.57 |
| Na | 7.71 | 7.55 | 7.85 |
| S | 10.73 | 10.51 | 11.09 |

1-28. (canceled)

29. Process for preparing ursodeoxycholic acid di-sodium 3,7-disulfate comprising:
a) reacting ursodeoxycholic acid with sulfamic acid in dimethylformamide to give ursodeoxycholic acid di-ammonium 3,7-disulfate;
b) treating the ursodeoxycholic acid di-ammonium 3,7-disulfate with organic sodium bases or inorganic sodium bases then treating the reaction mixture with an inorganic acid until a pH between 3.0 and 4.5 is reached to give ursodeoxycholic acid di-sodium 3,7-disulfate in solution.

30. Process as claimed in claim 29, wherein the reaction of stage a) is conducted at a temperature between 40° C. and 110° C.

31. Process as claimed in claim 30, wherein the temperature is between 80° C. and 90° C.

32. Process as claimed in claim 29, wherein ursodeoxycholic acid di-ammonium 3,7-disulfate is separated from the reaction mixture of stage a) by fractional crystallisation with acetone.

33. Process as claimed in claim 29, wherein the inorganic sodium bases in stage b) are selected from the group consisting of sodium hydroxide, sodium carbonate and sodium bicarbonate.

34. Process as claimed in claim 29, wherein the organic sodium bases are sodium acetate.

35. Process as claimed in claim 29, wherein in stage b) the treatment of ursodeoxycholic acid di-ammonium 3,7-disulfate with organic sodium bases or inorganic sodium bases is conducted in alcoholic solvent.

36. Process as claimed in claim 35, wherein the alcoholic solvent is selected from the group consisting of linear or branched lower C1-C4 alcohols or their mixtures.

37. Process as claimed in claim 36, wherein the alcohol is methanol.

38. Process as claimed in claim 29, wherein in stage b) the treatment with organic sodium bases or inorganic sodium bases is conducted at a temperature between −10° C. and 30° C.

39. Process as claimed in claim 38, wherein the temperature is between 0° C. and 5° C.

40. Process as claimed in claim 29, wherein in stage b) the treatment with organic sodium bases or inorganic sodium bases is conducted under vacuum.

41. Process as claimed in claim 29, wherein in stage b) the acidification of the reaction mixture after treatment with organic sodium bases or inorganic sodium bases is conducted by treating the reaction mass with an inorganic acid selected from the group consisting of hydrochloric acid, sulphuric acid, 85% (w/w) phosphoric acid or their mixtures.

42. Process as claimed in claim 41, wherein the acid is 85% phosphoric acid.

43. Process as claimed in claim 29, also comprising stage c), recovering ursodeoxycholic acid di-sodium 3,7-disulfate from the reaction mixture, said stage comprising: c′) removing, by filtration, precipitated inorganic salts formed after acidification treatment and c″) precipitating ursodeoxycholic acid di-sodium 3,7-disulfate from the filtrate whereby the solution containing ursodeoxycholic acid di-sodium 3,7-disulfate is concentrated by distillation and the residue is re-dissolved in organic solvent to isolate ursodeoxycholic acid di-sodium 3,7-disulfate.

44. Process as claimed in claim 41, wherein in stage c′) the filtration to remove the precipitated inorganic salts formed after acidification treatment is facilitated by treating the reaction mixture derived from stage b) with an organic solvent.

45. The process as claimed in claim 44, wherein said organic solvent is acetone.

46. Process as claimed in claim 43 wherein in stage c″) the residue obtained by concentrating the solution containing ursodeoxycholic acid di-sodium 3,7-disulfate by distillation is re-dissolved in an organic solvent, at a temperature between 20° C. and 70° C., and the suspension thus obtained is then cooled to room temperature and filtered to obtain ursodeoxycholic acid di-sodium 3,7-disulfate as precipitate.

47. The process as claimed in claim 46, wherein said organic solvent is acetone and ursodeoxycholic acid di-sodium 3,7 disulfate is dissolved in said solvent at a temperature comprised between 55° and 65° C.

48. Ursodeoxycholic acid di-ammonium 3,7-disulfate of formula:

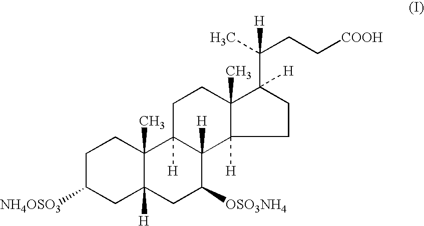

49. Process for synthesizing ursodeoxycholic acid di-ammonium 3,7-disulfate comprising reacting ursodeoxycholic acid with sulfamic acid in N,N-dimethylformamide.

50. Process as claimed in claim 49, wherein the reaction with sulfamic acid is conducted at a temperature between 40° C. and 110° C.

51. Process as claimed in claim 50, wherein the temperature is between 80° C. and 90° C.

52. Process as claimed in claim 49, wherein the ursodeoxycholic acid di-ammonium 3,7-disulfate is separated from the reaction mixture by fractional crystallisation with acetone.
